(19) United States
(12) Patent Application Publication
(10) Pub. No.: US 2021/0324441 A1
(43) Pub. Date: Oct. 21, 2021

(54) CHARACTERIZATION OF MICROORGANISMS VIA MALDI-TOF

(71) Applicant: BIOMÉRIEUX, Marcy L'Étoile (FR)

(72) Inventors: Mahendrasingh RAMJEET, Lyon (FR); Jérémy SURRE, Brignoud (FR); Audrey FERULLO, Marseille (FR)

(73) Assignee: BIOMÉRIEUX, Marcy L'Étoile (FR)

(21) Appl. No.: 17/359,752

(22) Filed: Jun. 28, 2021

Related U.S. Application Data
(63) Continuation of application No. 15/328,973, filed on Jan. 25, 2017.

(30) Foreign Application Priority Data
Jul. 30, 2014 (FR) 1457389

(51) Int. Cl. C12Q 1/24; C12Q 1/04; C12Q 1/18; G01N 33/68; G01N 1/36

(52) U.S. Cl. CPC C12Q 1/24; C12Q 1/04; G01N 1/36; G01N 33/6848; G01N 33/6851; C12Q 1/18

(57) ABSTRACT

A method of preparing a characterization zone of an analysis plate to carry out a characterization of a population of a microorganism in the presence of an antimicrobial agent by mass spectrometry using the MALDI technique. The method involves the following steps in succession:
a step for providing an analysis plate for a characterization by means of the MALDI technique, the plate including an analysis zone carrying an antimicrobial agent;
a step of depositing the population of a microorganism onto said analysis zone in contact with the antimicrobial agent;
an incubation step of preserving the analysis plate under conditions and for a sufficient time to allow the antimicrobial agent and the microorganism that is present to interact; and
a step of depositing a matrix that is suitable for the MALDI technique onto the analysis zone;
associated characterization and functionalization methods, analysis plates and uses.

This application is a continuation of U.S. application Ser. No. 15/328,973, filed Jan. 25, 2017, which is a 371 of International application number PCT/FR2015/052105, filed Jul. 29, 2015, which claims priority to French patent application number 1457389, filed Jul. 30, 2014, the contents of which are incorporated herein by reference.

The present invention relates to the field of microbiology. More precisely, the invention relates to characterizing a population of at least one microorganism using mass spectrometry, and in particular mass spectrometry (MS) by matrix-assisted desorption-ionization and time of flight known as MALDI-TOF.

For several years, the MALDI-TOF technique has been used to carry out rapid identification of microorganisms on the species level. Various types of instruments that are suitable for a characterization of this type are marketed by the Applicant and also in particular by Bruker Daltonics and Andromas.

A microorganism is identified from a MALDI-TOF spectrum of the most abundant proteins in the microorganism, by comparison with reference data in order to identify the family, genus, and usually the species of the microorganism. As a rule, the protocol employed comprises depositing at least a portion of a microorganism colony on a MALDI plate, adding a matrix suitable for the MALDI technique, acquiring the mass spectrum and identifying the species by comparison with reference data stored in a database.

More recently, the MALDI technique has also been used to detect resistance of a microorganism to an antibiotic, and in particular to identify a phenotype that is responsible for hydrolysis of beta-lactam type antibiotics, due to the secretion of beta-lactamase type enzymes, and in particular of the carbapenemase type. Applications US 2012/0196309 and WO 2012/023845 may be mentioned in this regard. Characterizing resistance by MALDI-TOF involves the detection of the hydrolyzed form and/or the loss of the native form of the beta-lactam antibiotic after incubation with said antibiotic of the sample that might contain a microorganism capable of producing a beta-lactamase.

Although the preparation of the sample to be deposited is not described in detail in those patent applications, in general it is envisaged that the microorganism is mixed with the antibiotic agent beforehand, and then the mixture is deposited on the MALDI plate or a lysate of microorganism(s) is used (see WO 2012/023845, in particular claim 15).

Document WO 2013/113699 envisages using mass spectrometry to evaluate the inhibiting effect of a substance on beta-lactamases, that evaluation possibly being carried out in the presence of bacteria.

The following publications: (Hrabak, Walkova et al. 2011, Hooff, van Kampen et al. 2012, Hrabak, Studentova et al. 2012, Sparbier, Schubert et al. 2012) provide a more detailed description of the protocols to be carried out for preparing samples, which protocols comprise the following steps:
preparing an inoculum comprising colonies of the microorganism;
centrifuging the inoculum;
re-suspending the bacterial pellet with a solution of antibiotic, with or without lysis reagent;
incubating for a period of 30 minutes to 3 hours;
centrifuging; and
transferring 1 microliter of supernatant onto a MALDI plate and adding 1 microliter of matrix, analysis by MALDI-TOF mass spectrometry.

Preparing the sample prior to detecting resistance by the MALDI-TOF technique is thus long and tedious. In fact, the procedure used to detect resistance by the MALDI-TOF technique necessitates preparing an inoculum with a high concentration and/or one or more centrifuging steps. Those steps require materials, consumables, and specific equipment and are thus consumers of consumables and time and operator expertise. In addition, prior preparation of that type makes it difficult to use the MALDI-TOF technique routinely for the detection of resistance, given that carrying out a characterization of a large number of samples per day cannot be envisaged. In fact, there is often a mismatch between the availability of the instrument to carry out the identification of the phenomenon of resistance by the MALDI-TOF technique and the availability of samples obtained after the obligatory preparation step. Another problem resides in the fact that the bacteria are incubated with the antibiotic in volumes of the order of 10 μL to 50 μL, which gives rise to a potentially high risk of reducing the enzyme-antibiotic interactions. As a consequence, there may be a reduction in the efficacy of the enzymatic activity, which would affect the sensitivity of detection and thus the robustness of the technique, in particular with microorganisms that are known to produce or secrete small quantities of enzymes.

The detection of resistance to an antimicrobial agent is also described in applications WO 2011/045544 and US 2012/0196309 as being capable of being carried out by using other mass spectrometry techniques such as mass spectrometry-mass spectrometry (MS-MS) and multiple reaction monitoring (MRM). Under such circumstances, analysis by mass spectrometry and thus ionization are not carried out on the microorganism, but on the proteins obtained after various purification operations.

In the context of the invention, the inventors propose implementing a novel method of preparing a characterization zone for characterizing a microorganism by the MALDI technique, which method can be used to identify a resistance to an antibiotic and is simple to implement. Furthermore, this novel method is compatible with obtaining various characterizations of the sample present in the characterization zone, at least one corresponding to identifying resistance to an antibiotic, and another possibly corresponding to identifying a microorganism.

The invention thus provides a method of characterizing a sample containing at least one microorganism using the MALDI-TOF technique, which can be used to discern whether a population of a microorganism that is present is or is not resistant to at least one antimicrobial agent, and within a relatively short time period, of the order of a few hours to one hour, or even less.

The invention also concerns a method of preparing a characterization zone of an analysis plate in order to carry out at least one characterization of a population of at least one microorganism in the presence of at least one antimicrobial agent, said characterization involving an analysis by mass spectrometry during which the population of at least one microorganism deposited on the analysis zone undergoes at least one ionization step by bombarding the analysis zone with a laser beam in accordance with the matrix-assisted laser desorption-ionization mass spectrometry technique known as MALDI;

the method of preparing the analysis zone being characterized in that it comprises the following steps in succession:
a step consisting in providing an analysis plate for a characterization by means of the MALDI technique, the plate comprising at least one analysis zone carrying an antimicrobial agent;
a step of depositing the population of at least one microorganism onto said analysis zone in contact with the antimicrobial agent;
an incubation step, consisting in preserving the analysis plate under conditions and for a sufficient time to allow the antimicrobial agent and the microorganism that is present to interact; and
a step of depositing a matrix that is suitable for the MALDI technique onto the analysis zone.

Preferably, in the context of the preparation process in accordance with the invention, a population of a single microorganism to be characterized is deposited. Thus, the characterization zone may then be used for characterizing a single microorganism.

In accordance with the invention, the deposited population of microorganism(s) is prepared without a prior step of contact with an antimicrobial agent, i.e. the deposited population of microorganism(s) is not mixed with the antimicrobial agent present on the characterization zone prior to being deposited.

The method in accordance with the invention is advantageously carried out on a population of microorganism(s) obtained after a step of concentration, enrichment and/or purification and/or corresponding to a colony or to a fraction of a colony obtained after growth on a suitable medium, in particular a gel medium.

By way of example, in the context of the invention, the antimicrobial agent is selected in a manner such as to make it possible to identify the resistance due to the production of beta-lactamase, and in particular of carbapenemase.

The antimicrobial agent is usually an antibiotic, preferably selected from penicillins, cephalosporins, cephamycins, carbapenems, and monobactams, and in particular from ampicillin, amoxicillin, ticarcillin, piperacillin, cefalotin, cefuroxime, cefoxitin, cefixime, cefotaxime, ceftazidime, ceftriaxone, cefpodoxime, cefepime, aztreonam, ertapenem, imipenem, meropenem, and faropenem.

The preparation method in accordance with the invention may comprise a functionalization step in order to obtain the analysis plate for a characterization by means of the MALDI technique comprising at least one analysis zone carrying an antimicrobial agent termed a functionalized zone, said functionalization step preferably being carried out by depositing an aqueous solution of the antimicrobial agent, followed by drying.

The invention also pertains to a method of characterizing a population of at least one microorganism, the characterization comprising at least determining the presence or otherwise of a population of a microorganism that is resistant to at least one antimicrobial agent;

the method being characterized in that it comprises the following steps in succession:
preparing at least one characterization zone of an analysis plate in accordance with a preparation method of the invention;
using matrix-assisted laser desorption-ionization time of flight mass spectrometry known as MALDI-TOF to analyse, at least once, a population of a microorganism deposited on the characterization zone, in order to be able to conclude whether a population of a microorganism that is resistant to the antimicrobial agent is present on the characterization zone.

In particular, the mass spectrometric analysis by means of the MALDI-TOF technique for concluding whether a population of a microorganism that is resistant to the antimicrobial agent is present on the characterization zone, consists in verifying the presence of the antimicrobial agent and/or a degradation product of the antimicrobial agent.

Advantageously, the characterization method in accordance with the invention comprises at least two characterizations. In particular, the characterization additionally comprises identifying the family, genus, or, preferably, the species of a population of a microorganism deposited on the characterization zone.

Preferably, the characterization method in accordance with the invention comprises:
identifying the family, genus, or, preferably, the species of a population of a microorganism deposited on the characterization zone by carrying out a first analysis by MALDI type mass spectrometry corresponding to a first step of ionizing a characterization zone, and using a first calibration for the analysis; and
determining the possible presence on the characterization zone of a population of a microorganism that is resistant to the antimicrobial agent by caring out a second analysis by mass spectrometry using the MALDI-TOF technique corresponding to a second step of ionizing the same characterization zone, and using a second calibration for the analysis.

Under such circumstances, identification of the family, genus, or, preferably, the species of a population of a microorganism, and determining the possible presence of the antimicrobial agent preferably concern the same microorganism.

The invention also provides a method of characterizing a population of at least one microorganism, the characterization comprising at least:
identifying the family, genus, or, preferably, the species of a population of a microorganism, by carrying out a first analysis by mass spectrometry using the MALDI technique corresponding to a first step of ionizing a characterization zone comprising the population of microorganism(s), the antimicrobial agent and a matrix suitable for the MALDI technique;
determining the presence of a population of a microorganism that is resistant to at least one antimicrobial agent, by carrying out a second analysis by mass spectrometry using the MALDI technique corresponding to a second step of ionizing a characterization zone comprising the population of at least one microorganism, the antimicrobial agent and a matrix suitable for the MALDI technique;

the method being characterized in that:
the first analysis and the second analysis are respectively carried out by means of a distinct first step and a distinct second step of ionizing the same characterization zone comprising the population of at least one microorganism and the antimicrobial agent;
the first analysis uses a first calibration, and the second analysis uses a second calibration that is different from the first calibration.

Preferably, the population that is deposited comprises a single microorganism to be characterized.

A characterization method of this type preferably employs a method of preparing a characterization zone of an analysis plate in accordance with the invention.

In the context of the invention, the same characterization zone and thus the same population of microorganism(s) undergoes two ionizations in succession in order to obtain the two desired characterizations: characterizing a phenomenon of resistance of a microorganism to an antimicrobial agent, and identification of the microorganism. The invention also provides a method of functionalizing an analysis zone of an analysis plate adapted to the MALDI technique, the method of functionalizing the analysis zone being characterized in that it comprises:
a step of functionalizing the analysis zone to make the analysis zone carry an antimicrobial agent.

The functionalizing step may be implemented by depositing an aqueous solution of the antimicrobial agent, followed by drying.

The invention also provides an analysis plate intended to receive a population of at least one microorganism to be characterized on an analysis zone by mass spectrometry in accordance with the MALDI technique, the plate being characterized in that it includes an antimicrobial agent deposited, in particular in the form of a solid deposit, on said analysis zone of the analysis plate, forming an analysis zone that is said to be functionalized. In particular, in an analysis plate of this type, the functionalized analysis zone(s) carrying the antimicrobial agent does/do not include a microorganism in a quantity that is detectable by the MALDI technique and/or the functionalized analysis zone(s) carrying the antimicrobial agent is/are dried.

The term “dried” in particular means that the antimicrobial agent is not in solution, but in the form of a solid deposit that adheres to the analysis zone onto which it has been deposited. In particular, maintaining an antimicrobial agent on an analysis zone that is said to be functionalized could be accomplished by electrostatic bonding, ionic bonding, covalent bonding, affinity bonding or in fact by means of an adhesive agent. In particular, the adhesive agent may be a polymer that is soluble in water. An example of an adhesive agent that could be used to immobilize the antimicrobial agent on the analysis zone or zones that may be mentioned is heptakis(2,6-di-O-methyl)-β-cyclodextrin.

In a particular embodiment, in an analysis plate intended to receive a population of at least one microorganism to be characterized, the or each functionalized analysis zone carries a single antimicrobial agent.

The analysis plate for receiving a population of at least one microorganism in accordance with the invention could be packaged in a hermetically sealed package. In particular, the hermetically sealed packaging could be suitable for protecting the plate from light and from moisture.

The functionalized analysis zone(s) could be obtained by depositing an aqueous solution of the antimicrobial agent, followed by drying.

Plates of this type could include a plurality of functionalized analysis zones, each carrying an antimicrobial agent, in particular with at least a first functionalized analysis zone carrying a first antimicrobial agent and a second functionalized analysis zone, which is distinct from the first zone, carrying a second antimicrobial agent that is distinct from the first antimicrobial agent.

Usually, plates of this type include at least one reference analysis zone intended to subsequently receive a population of a reference microorganism, and in that the surface of the reference analysis zone is free from antimicrobial agent.

The invention also provides the use of an analysis plate in accordance with the invention in a characterization method in accordance with the invention.

The description below, made with reference to the accompanying drawings, contributes to a better understanding of the invention.

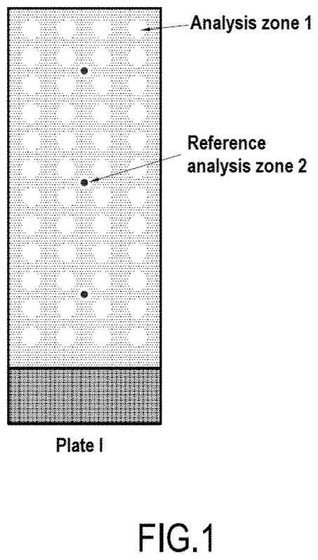

FIG. 1 is a schematic top view of a MALDI plate.

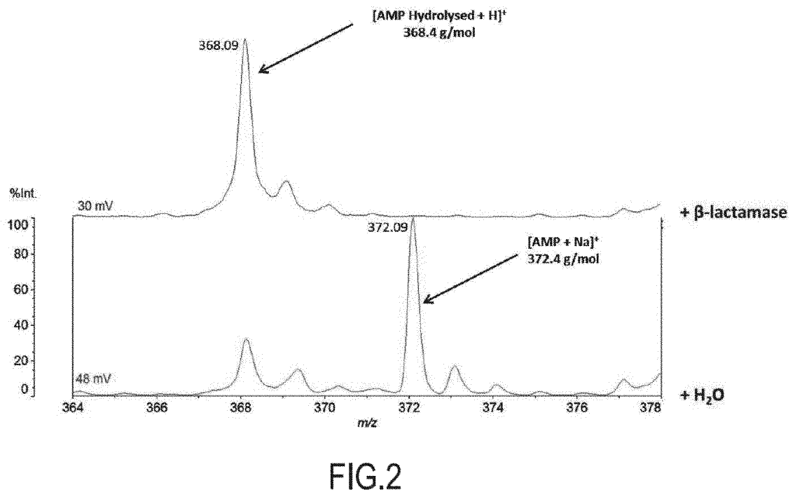

FIGS. 2 to 4 are mass spectra obtained in the examples by means of the MALDI-TOF technique. In these figures, the intensity scale is a relative scale by reference to the most intense peak of the spectrum. As an example, over a selected range of mass (in particular 200 m/z to 1200 m/z), if the most intense peak is 100 mV, it is denoted as 100% (as mentioned on the left hand side of the spectra). The less intense peaks are denoted relative to the most intense peak: thus, a peak with an intensity of 75 mV reaches 75% of the scale (on the spectrum containing the maximum intensity peak of 100 mV). As a consequence, for the same spectrum, the level of intensity of the peaks between the strains cannot be compared. In contrast, these spectra can be used, for one and the same strain, to compare the intensity between the native peak of the antimicrobial agent and that of its degradation product. It is also possible, for one and the same strain, to compare the intensity between the native or hydrolyzed peaks and a control peak that has not been submitted to variations induced by the biological/enzymatic activity of the microorganism to be tested. The following peaks: a peak of the HCCA matrix, the peak of a peptide or of a reference molecule added to the matrix or that has already been dried onto the analysis zone, could be considered as control peaks.

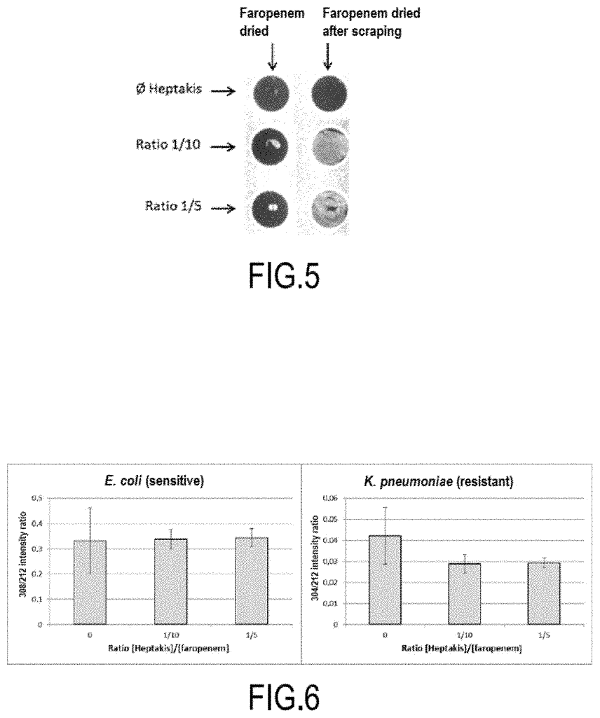

FIG. 5 shows the appearance of analysis zones (spots) onto which an antibiotic, faropenem, has been deposited, in the presence and in the absence of adhesive agent (heptakis), before and after scraping with an inoculation loop.

FIG. 6 shows the variation in the ratios of the intensities of the peaks of native faropenem and hydrolyzed faropenem, obtained by the MALDI-TOF technique compared with a control peak of HCCA as a function of the [Heptakis]/[faropenem] ratios.

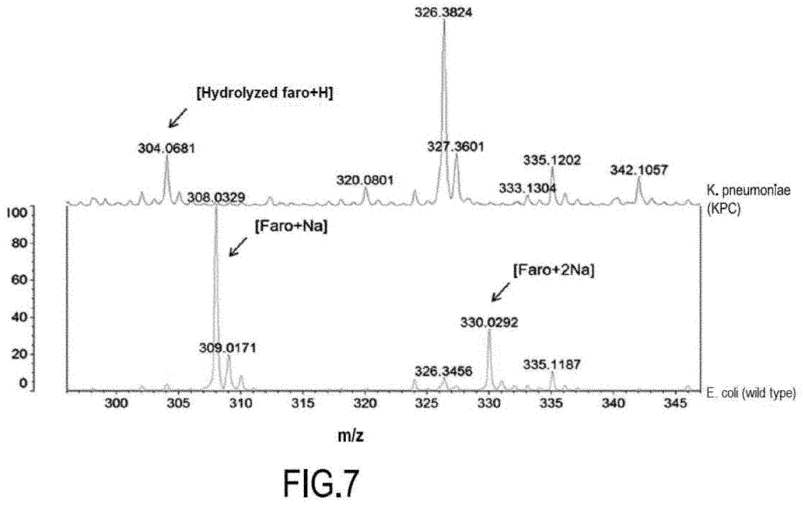

FIG. 7 presents the mass spectra obtained during the second series of acquisitions of Example 5.

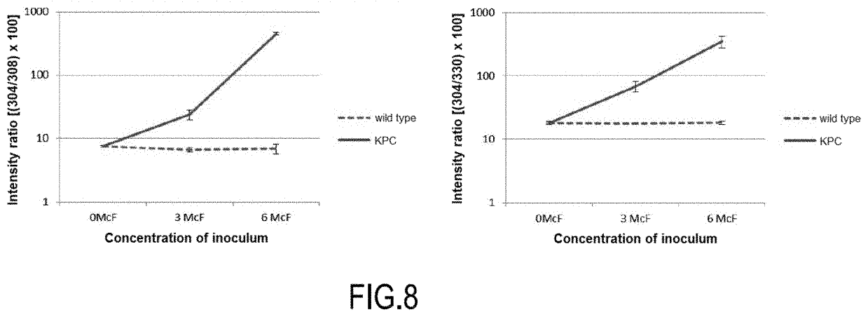

FIG. 8 shows the variation in the ratios of the 304/308 and 304/330 intensities as a function of the concentration of inoculum used for the two strains tested in Example 5.

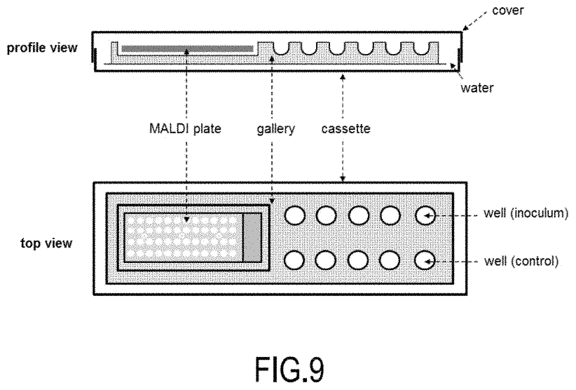

FIG. 9 represents an embodiment of a case integrating a MALDI plate that could be adapted to depositing a population of microorganisms in the liquid form.

### MALDI ANALYSIS PLATES

A MALDI analysis plate has at least one, and in general a plurality of analysis zones. The analysis zones are in the form of spots, usually circular in shape. In order to promote subsequent ionization, at least at the level of the analysis zone or zones, the surface of the plate is conductive. By way of example, an analysis plate of this type is formed by a polymer such as polypropylene, said polymer being coated with a layer of stainless steel. The polymer may contain a conductive material such as carbon black. By way of example, such a plate may be a plate marketed by Shimadzu, with the reference “Fleximass™ DS disposable MALDI targets”.

A variety of MALDI plates are commercially available, such as Fleximass DS plates from bioMérieux (disposable or reusable) and Maldi Biotarget plates from Bruker Daltonics. Plates I of this type usually comprise 48 to 96 analysis zones 1 or spots, and at least one, or even two or three reference analysis zones 2 the sizes of which, as shown in the example of FIG. 1, differ from that of the analysis zones.

In the context of the invention, the term “characterization zone” is used for an analysis zone carrying an antimicrobial agent, a population of microorganism(s) and a matrix that is suitable for the MALDI technique.

### Functionalizing the Analysis Zone

The term “antimicrobial agent” means a compound that is capable of reducing the viability of a microorganism and/or of reducing its growth or reproduction. Antimicrobial agents of this type may be antibiotics when they are directed against bacteria. However, the invention is of application to any type of microorganism of the bacterial, yeast, mold, or parasite type, and thus to the corresponding antimicrobial agents.

Preferably, the antimicrobial agent is an antibody such as a beta-lactam, in particular selected from penicillins, cephalosporins, cephamycins, carbapenems, and monobactams, and in particular from ampicillin, amoxicillin, ticarcillin, piperacillin, cefalotin, cefuroxime, cefoxitin, cefixime, cefotaxime, ceftazidime, ceftriaxone, cefpodoxime, cefepime, aztreonam, ertapenem, imipenem, meropenem, and faropenem.

Carbapenemes are used in particular as a last resort to combat Gram-negative bacteria such as the enterobacterium family, Pseudomonas and Acinetobacter. An antibiotic of this type is thus deposited on the analysis zone when it is suspected that the microorganism that is present is an enterobacterium or another Gram-negative species that might have resistance to carbapenemes.

Preferably, it is deposited from an aqueous solution of the antimicrobial agent.

A buffer adapted to the solubility of the antimicrobial agent as well as to an optimized activity of the mechanism at the origin of the targeted resistance could also be used to prepare the solution of antimicrobial agent. Adding zinc salts (in particular of the zinc chloride or sulfate type) to the antimicrobial solution could also be envisaged for an optimized activity of the metallo-beta-lactamases.

A droplet, e.g. approximately of 1 to 2 microliters, of the antimicrobial solution could be deposited in a manner such that the whole of the analysis zone is covered. The water contained in the solution is then evaporated off, for example simply by drying in ambient air and at ambient temperature. By way of example, the analysis plate may be left at a temperature that is in the range 17° C. to 40° C., and in particular at ambient temperature (22° C.). It is also possible to transfer it to a thermostatted chamber, e.g. at 37° C., in order to accelerate drying.

The antimicrobial agent is deposited in aqueous solution in very simple manner, this deposition being followed by a drying operation. Thus, an analysis zone is obtained that carries an antimicrobial agent that is said to be functionalized. It is also possible for the antimicrobial agent to be immobilized on the analysis zone by electrostatic, ionic, covalent, or affinity bonding, or by means of an adhesive agent. Simply depositing the antimicrobial agent would not provide satisfactory immobilization thereof. Specifically, if the antimicrobial agent does not adhere sufficiently to the characterization zone, this can result in a loss of antimicrobial agent when depositing the population of microorganism(s), which would then require a certain amount of dexterity on the part of the operator when producing the deposit, or indeed it can result in a loss of antimicrobial agent by detachment of the deposit during storage of the MALDI plate for subsequent use. In addition, in place of a simple deposit, the antimicrobial agent could be linked to the analysis zone via electrostatic, ionic, or covalent bonds with or without the use of an optionally-specific linker or arm (antibody, recombinant phage proteins), by using the interaction of biotin/streptavidin already grafted to the surface of the analysis zone and to the antimicrobial agent, or by any other type of bond adapted to the nature of the antimicrobial agent and to the surface of the analysis zone, or indeed by means of an adhesive agent. However, the mode of bonding or of depositing should be selected in a manner such that any interaction of the antimicrobial agent with a microorganism is not compromised, since that could result in masking a resistance phenomenon. In particular, it is preferable to immobilize the antimicrobial agent by using an adhesive agent rather than by covalent bonding or affinity bonding, in order to prevent changes to the conformation of the antimicrobial agent and to ensure that it can gain proper access to the active site of the enzyme that could be generated by the microorganism.

When an adhesive agent is used, i.e. an agent that adheres to the plate and thus improves immobilization of the antimicrobial agent thereto, then a mixture of the adhesive agent and the antimicrobial agent in aqueous solution is deposited. In particular, the adhesive agent may be a polymer that is soluble in water. An example that may be mentioned of an adhesive agent that can be used to immobilize the antimicrobial agent to the analysis zone or zones is heptakis(2,6-di-O-methyl)-β-cyclodextrin. The adhesive agent should be selected as a function of the antimicrobial agent to be immobilized on the analysis zone. In particular, it should be selected as a function of its mass, in a manner such that its presence does not distort subsequent MALDI detection aimed at determining the presence or absence of the antimicrobial agent that is present and/or of its degradation products. The person skilled in the art should adjust the quantity of adhesive agent used, which must not be too high in order to ensure that the antimicrobial agent is accessible to the microorganism population when the latter has been deposited. As an example, with heptakis(2,6-di-O-methyl)-β-cyclodextrin, it is possible to select a ratio by weight of heptakis(2,6-di-O-methyl)-β-cyclodextrin divided by antimicrobial agent from 1/20 to 1/2, preferably from 1/10 to 1/5.

The person skilled in the art should adapt the quantity of antimicrobial agent deposited on an analysis zone as a function of the antimicrobial agent in question. In fact, depending on the degree of ionization of the molecule, a sufficient quantity must be deposited in order to be able to detect the peak(s) corresponding to the antimicrobial agent in MALDI-TOF with intensities above the background noise. In contrast, too large a quantity of antimicrobial agent runs the risk of masking detection of the phenomenon at the origin of the resistance that is to be characterized, such that the reduction in the intensity of the peak corresponding to the native antimicrobial agent cannot be observed. The excess antimicrobial agent could in particular compromise detecting beta-lactamases with low activity. As an example, 0.04 g/m² to 4 g/m² of antimicrobial agent should be deposited. To this end, a solution of antimicrobial agent in water, in particular in ultra-pure water, should be deposited at a concentration from 0.1 mg/mL to 10 mg/mL. By way of example, for ampicillin, an aqueous solution comprising 1.7 mg/mL to 10 mg/mL of ampicillin could be deposited, and for faropenem, an aqueous solution comprising 0.1 mg/mL to 1 mg/mL of faropenem could be deposited.

A characterization zone should preferably carry a single antimicrobial agent, although the use of a plurality of antimicrobial agents on one and the same analysis zone is not excluded. A characterization zone carrying a plurality of antimicrobial agents could be used to test for the presence of a plurality of enzymes at the same time, and thus for different resistance phenomena. When a characterization zone carrying a plurality of antimicrobial agents is used, the agents should be selected in a manner such that the masses of their native form and/or their degradation products under the action of the target enzyme do not overlap, so that they can be detected separately by MALDI-TOF. As an example, it would be possible to deposit another antimicrobial agent from the same family or from a different family in addition to a first antimicrobial agent. As an example, certain carbapenemes are more adapted to revealing a particular carbapenemase. Thus, it is possible to envisage having a plurality of types of carbapenemes or other beta-lactams on one and the same characterization zone. In contrast, if two antimicrobial agents are deposited on one and the same zone, they should be selected in a manner such that their spectra of activity do not interfere with each other and that they can be detected distinctly by MALDI-TOF.

It is also possible to deposit another compound in addition to the antimicrobial agent. With beta-lactams, it is also possible to deposit a beta-lactamase inhibitor in order to characterize an ESBL phenomenon (extended spectrum beta-lactamase), as employed in particular in the application WO 2012/023845. In particular, a combination of a beta-lactam with a beta-lactamase inhibitor such as clavulanic acid, sulbactam or razobactam could be deposited. When the MALDI plate comprises a plurality of analysis zones, at least two zones or even more advantageously carry a different antimicrobial agent. It is then possible to characterize several populations of microorganism(s) with a single plate. Each of the zones could carry a different antimicrobial agent. Usually, however, the characterizations are carried out in duplicate, such that a single antimicrobial agent is present on at least two analysis zones or even on more.

Functionalized MALDI plates of this type may be supplied directly to the consumer who would then only have to deposit the population of microorganism(s) to be studied and then, after an incubation step, the MALDI matrix. They may be marketed as individual packs or in packs comprising several plates.

### Preparing and Depositing the Population of Microorganism(s)

In the context of the invention, a population of microorganism(s) is deposited on an analysis zone of a MALDI plate functionalized with an antimicrobial agent in order subsequently to proceed with characterizing it.

The population of microorganism(s) may originate from a variety of sources. Examples of sources of microorganism(s) that may be mentioned are samples of biological origin, in particular of animal or human origin. A sample of this type may correspond to a biological fluid sample, of the whole blood, serum, plasma, urine, cerebrospinal, or organic secretion type, or a tissue sample, or isolated cells. This sample may be deposited as is or, as is preferable, it may undergo preparation of type comprising enrichment or culture concentration and/or extraction, or a purification step using methods known to the person skilled in the art, prior to being deposited onto the analysis zone under consideration. However, a preparation of that type must not be of the type corresponding to a lysis step that would cause disintegration of the microorganisms and loss of their content before being deposited on the analysis zone. The population of microorganism(s) could be deposited in the form of an inoculum. In the context of the invention, the population of microorganism(s) deposited on the analysis zone is preferably a population of live microorganism(s), although extracting the population of microorganism(s) from a biological sample using a detergent that might affect viability is not excluded. However, in such circumstances, in order to carry out an immediate test of the population of microorganism(s) using MALDI, the stock of active enzymes already present could be used to characterize the population of microorganism(s) by detecting any phenomenon of resistance.

The source of the population of microorganism(s) may also be an agrifood product such as meat, milk, yogurt, and any other consumable product that might become contaminated, or indeed a cosmetic or a pharmaceutical product. Here again, a product of this type might be subjected to an enrichment or culture type preparation, a concentration, and/or an extraction or purification step in order to obtain the population of microorganism(s) to be deposited.

Usually, the source of microorganism(s) may previously be placed under culture in a broth or on a gel so as to enrich it in microorganisms. Gel or broth media of this type are well known to the person skilled in the art. Enrichment on gel is particularly favored, because it can be used to obtain colonies of microorganisms that can be deposited directly onto the analysis zone.

In the context of the invention, it is preferable to deposit on the analysis zone a cellular medium comprising a bacterial population rather than one or more proteins obtained after an extraction or purification step, as with MS/MS or MRM techniques. Preferably, the deposited population of microorganisms contains at least 10⁵ cfu of microorganisms. By way of example, 10⁵ cfu to 10⁹ cfu of a microorganism could be deposited. As an example, it is possible to proceed directly to depositing a biomass, a drop of a suspension of microorganisms in ultra-pure water or a buffer. A colony or a fraction of a microorganism colony could be deposited.

The deposited population preferably comprises a single species of microorganism. However, depositing a population comprising different microorganisms onto the analysis zone is not excluded. In this case, it is preferable for the microorganisms to be known for developing different resistance mechanisms so as to be able to know which microorganism presents the resistance that is identified.

In the context of the invention, it is not useful to carry out a particular preparation of a sample that is to be deposited. In particular, the population of microorganism(s) is deposited without previously being put into contact with an antimicrobial agent. In fact, in the context of the invention, it is not necessary to carry out lengthy and tedious preparation of a sample to be deposited; the population of microorganism(s) that is deposited can be prepared without a centrifuging step.

Deposition is carried out in a manner such that the population of microorganism(s) is deposited onto the analysis zone in a uniform manner. For this purpose, it is possible to use the procedures described for carrying out standard identification of microorganisms as appear in the instruction manuals for commercial MALDI-TOF instruments, such as the VITEK® MS instrument marketed by bioMérieux.

However, in addition to the population of microorganism(s), it is also possible to add a compound that is known to accelerate the enzymatic reaction occurring in the resistance mechanism under consideration. That compound may be zinc, for example, in the form of ZnCl₂ or zinc sulfate, in particular, which is an important co-factor in the activity of metallo-beta-lactamases. That compound may already have been deposited on the analysis zone in combination with the antimicrobial agent or at any other time during the preparation of the characterization zone.

The fact that the deposit on the analysis zone does in fact contain a population of at least one microorganism can be determined initially by means of a suitable test, in particular by the fact that it is a colony isolated on gel. Preferably, a single population of a single microorganism is deposited on the analysis zone.

### Incubation

After depositing the population of microorganism(s), the analysis zone carrying both the antimicrobial agent and the population of a microorganism to be characterized is subjected to an incubation step in order to allow the microorganism and the antimicrobial agent to interact and thus, when in the presence of a population of microorganisms that is resistant to the antimicrobial agent, to allow the reaction/phenomenon at the origin of the resistance to occur. In particular, when the resistance phenomenon to be detected is due to the presence of an enzyme produced by the deposited microorganism, incubation may be carried out in a manner such as to allow the enzymatic reaction to occur.

In the context of the invention, the phenomenon responsible for the resistance to an antimicrobial agent thus occurs directly on the MALDI plate and not at all prior to depositing the population of microorganism(s) already in the presence of an antimicrobial agent on the MALDI plate, as happens with prior art techniques. The phenomenon responsible for the resistance to an antimicrobial agent occurs in a minimum volume corresponding to the characterization zone. Thus, problems with dilution encountered with prior art techniques are limited.

The incubation conditions and period should be adapted by the person skilled in the art as a function of the resistance phenomenon to be characterized.

By way of example, the analysis plate may be left at a temperature that is in the range 17° C. to 40° C., and in particular at ambient temperature (22° C.). It is also possible to transfer it to a thermostatted chamber, for example at 37° C., in order to promote the enzymatic reaction that might be at the origin of the resistance phenomenon.

Humidity conditions should be adapted to prevent the microorganisms present from drying out, in particular when the resistance phenomenon to be detected employs a hydrolysis reaction. The plate could be placed in a moist atmosphere during incubation, at least so that the amount of moisture provided directly by the deposit containing the population of microorganism(s) is sufficient.

Under the selected conditions, the incubation time should be sufficient to allow subsequent detection, by MS MALDI-TOF, of the phenomenon of resistance that is to be detected, in particular enzymatic reaction for resistance phenomena mediated by an enzyme. Incubation is usually carried out for at least 5 minutes, more preferably for at least 20 minutes, and yet more preferably for a period of 45 to 90 minutes.

In the context of the invention, it has been shown that carrying out an incubation step of this type does not in any way have a deleterious effect on subsequent identification of the microorganism if such a characterization is to be carried out in addition to detection of the phenomenon of resistance.

### Depositing the MALDI Matrix

In general, the matrices used in the MALDI technique are photosensitive and crystallize in the presence of the population of microorganism(s), while preserving the integrity of the molecules and microorganisms present. Matrices of this type, in particular suitable for the MALDI-TOF MS technique, are well known and, for example, constituted from a compound selected from: 3,5-dimethoxy-4-hydroxycinnamic acid, α-cyano-4-hydroxycinnamic acid, ferulic acid, and 2,5-dihydroxybenzoic acid. Many other compounds are known to the person skilled in the art. There are also liquid matrices that do not crystallize either under atmospheric pressure or when under pressure. Any other compound that could be used to ionize the molecules present in the characterization zone under the effect of a laser beam could be used.

In order to produce the matrix, a compound of this type is dissolved, usually in water, preferably of an “ultra-pure” quality, or in a mixture of water and organic solvent(s). Examples of organic solvents that are in conventional use and that may be mentioned are acetone, acetonitrile, methanol, and ethanol. Trifluoroacetic acid (TFA) can sometimes be added. By way of example, one example of a matrix is constituted by 20 mg/mL of sinapic acid in an acetonitrile/water/TFA mixture of 50/50/0.1 (v/v)). The organic solvent allows the hybdrophobic molecules present to dissolve in solution, while the water can be used to dissolve the hydrophilic molecules. The presence of acid such as TFA encourages ionization of the molecules by proton capture (H⁺).

The solution constituting the matrix is deposited directly onto the analysis zone and then covers the population of microorganisms and the antimicrobial agent present thereon.

Optionally, the method in accordance with the invention may also contain a step of crystallizing the matrix that is present before the step of ionizing the characterization zone. Usually, the matrix is crystallized by allowing the matrix to dry in ambient air. The solvent present in the matrix is thus evaporated off, for example, by leaving the analysis plate at a temperature that is, for example, in the range from 17° C. to 30° C., and in particular at ambient temperature (22° C.) for several minutes, for example for 5 minutes to 2 hours. This evaporation of the solvent allows the matrix in which the population of microorganism(s) and the antimicrobial agent are distributed to crystallize.

### Ionization and Obtaining the Mass Spectrum

The population of microorganism(s) and the antimicrobial agent, placed in the MALDI matrix and forming the characterization zone are subjected to soft ionization.

The laser beam used for ionization may have any wavelength that is favorable to sublimating or to vaporizing the matrix. Preferably, an ultraviolet wavelength or even an infrared wavelength is used. By way of example, this ionization may be carried out with a nitrogen laser emitting ultraviolet (UV) radiation at 337.1 nm.

During ionization, the population of microorganism(s) and the antimicrobial agent are subjected to laser excitation. The matrix then absorbs the light energy, and restitution of that energy causes the matrix to sublime, causes the molecules present in the population of microorganism(s) and in the antimicrobial agent to be desorbed, and causes material to appear in a state that is termed a plasma. In that plasma, charges are exchanged between molecules of the matrix, of the microorganisms, and of the antimicrobial agent. As an example, protons could be torn from the matrix and transferred to proteins, peptides, and organic compounds present in the characterization zone. This step can be used to carry out soft ionization of the molecules present without inducing their destruction. The population of microorganism(s) and the antimicrobial agent then release ions of different sizes. These ions are then accelerated by an electrical field and fly freely in a tube under reduced pressure, known as the flight tube. The pressure applied during ionization and during acceleration of the ions generated is usually in the range 10⁻⁶ to 10⁻⁹ millibar [mbar]. The smallest ions then “fly” faster than the larger ions, thereby allowing them to be separated. A detector is situated at the terminal end of the flight tube. The times of flight (TOF) of the ions is used to calculate their masses. Thus, a mass spectrum is obtained that represents the intensity of the signal corresponding to the number of molecules ionized for the same mass per charge (m/z), as a function of the m/z ratio of the molecules that strike the detector. The mass-to-charge ratio (m/z) is expressed in Thomsons [Th]. Once introduced into the mass spectrometer, the spectrum of a characterization zone is obtained very rapidly, usually in less than a minute.

A method of MALDI-TOF mass spectrometry suitable for use in accordance with the invention may in particular comprise the following steps in succession in order to obtain the mass spectrum:
providing a characterization zone comprising the population of microorganism(s) to be studied and at least one antimicrobial agent in a matrix adapted for MALDI spectrometry;
optionally, carrying out crystallization of the matrix in which the population of microorganism(s) and the antimicrobial agent are disposed;
ionizing the mixture of the population of microorganism(s) and the antimicrobial agent, and the matrix using a laser beam;
accelerating the ionized molecules obtained by means of a potential difference;
allowing the ionized and accelerated molecules to move freely in a tube under reduced pressure;
detecting at least a portion of the ionized molecules at the outlet from the tube in a manner such as to measure the time they have taken to pass through the tube under reduced pressure and to obtain a signal corresponding to the number of ionized molecules reaching the detector at a given time; and
calculating the mass-to-charge ratio (m/z) of the detected molecules in a manner such as to obtain a signal corresponding to the number of ionized molecules with the same mass-to-charge (m/z) as a function of the ratio m/z of the detected molecules.

In general, the ratio m/z is calculated by taking into account an initial calibration of the mass spectrometer employed in the form of an equation linking the mass-to-charge ratio (m/z) and the time of flight of the ionized molecules in the reduced pressure tube.

Calibration consists in using a molecule or a microorganism (depending on the characterization) that provides ionized molecules covering the range of masses corresponding to the envisaged characterization. The m/z ratios of these ionized molecules act as standards in order to allow the instrument to measure the masses appropriately.

To identify the microorganism, the calibration could be carried out using a strain of bacteria with ionized molecules having m/z ratios covering the range of masses used for identification (typically in the range 2000 Daltons (Da) to 20000 Da for yeasts, molds, bacteria, or parasites). In order to detect the signals corresponding to the antimicrobial agent, the calibration can be carried out using a mixture of peptides of small masses. In the context of the invention, the calibrant pepMIX 6 (LaserBio Labs) covering a range of masses of 350 Da to 1000 Da could be used, for example.

Any type of MALDI-TOF mass spectrometer could be used to produce the mass spectrum. Spectrometers of this type comprise:

i) a source of ionization (in general a UV laser) for ionizing the mixture of the population of microorganism(s) and the antimicrobial agent, and the matrix;

ii) an ionized molecule accelerator, applying a potential difference;

iii) a reduced pressure tube in which the ionized and accelerated molecules move;

iv) a mass analyzer intended to separate the molecular ions formed as a function of their mass-to-charge ratio (m/z); and

v) a detector intended to measure the signal produced directly by the molecular ions.

In the context of the invention, analysis by MALDI-TOF is preferably a simple MALDI-TOF analysis, although analysis by MALDI-TOF TOF is not excluded. Analysis by MALDI-TOF-TOF, although more complex, could be envisaged in particular, in order to improve the sensitivity of detection in certain circumstances, and it requires an instrument that is suitable for analysis of this type.

### Detection of Resistance to an Antimicrobial Agent

The term “resistance” means a phenomenon in which a microorganism does not exhibit a reduction in its viability or a reduction in its growth or in its reproduction when it is exposed to a concentration of an antimicrobial agent that is recognized as being effective against said microorganism in the absence of resistance.

A resistance mechanism may be identified from the mass spectrum obtained for a characterization zone under consideration by detecting, on the mass resulting spectrum, of a peak with a given mass or of a change in the mass peak compared with a reference mass spectrum, in particular compared with a mass spectrum of the antimicrobial agent present in said characterization zone.

In the context of the invention, it has been demonstrated that carrying out mass spectrometry by MALDI-TOF directly on a microorganism in the presence of an antimicrobial agent should allow molecules of interest that are pertinent to the determination of a resistance to said antimicrobial agent to be detected. The determination of any resistance of a population of a microorganism may thus comprise the following steps:

a1) providing a reference mass spectrum, for example for the antimicrobial agent and/or for its degradation products; degradation products of this type are the result of the resistance phenomenon and are due, in particular, to the presence of a degradation enzyme;

b1) applying ionization to the population of microorganism(s) and the antimicrobial agent deposited on the analysis zone and brought into the presence of the matrix (corresponding to a characterization zone in accordance with the invention);

c1) acquiring a mass spectrum obtained following this ionization, in the range of masses of interest for determining the resistance; and

d1) comparing the mass spectrum obtained in step c1) with the reference spectrum and deducing therefrom the presence or otherwise of a resistance.

In step d1) for example, if, on the mass spectrum obtained in step c1), the peak or peaks with a characteristic mass for the antimicrobial agent have disappeared and/or if one or more of the peak(s) with characteristic mass(es) of one or more degradation products of the antimicrobial agent is(are) present, then it can be deduced that a microorganism that is resistant to the antimicrobial agent is present. By way of example, the interpretation could be carried out from the ratio of intensities between a peak with a characteristic mass for the antimicrobial agent or one of its degradation products and a peak with a characteristic mass for an external calibrant, or from the ratio of intensities between a peak with a characteristic mass for the antimicrobial agent and a peak with a characteristic mass for a degradation product of the antimicrobial agent.

It is also possible to compare the level of intensity between the peak or peaks with a characteristic mass for the antimicrobial agent or the peak or peaks with a characteristic mass for one or more degradation products of the antimicrobial agent and one or more reference peaks of a compound that is present and that has not been subjected to the variations induced by the biological/enzymatic activity to be tested. Examples of reference peaks that may be considered are one or more peaks of the MALDI matrix, one or more peaks of a peptide or a reference molecule added to the matrix or that has already been dried on the analysis zone, or one or more peaks that correspond to a molecule of the microorganism present (for example a metabolite) that is always present and invariable in several species.

When determining resistance, a calibration is carried out in the range of masses corresponding to low masses, typically in the range 200 Da to 1200 Da, and preferably in the range 200 Da to 600 Da. The mass spectrum obtained in step c1) is also included within this range of masses. In order to carry out this calibration, two microliters of a calibrating solution composed of a mixture of peptides (pepMIX6, LaserBio Labs) and of HCCA matrix, α-cyano-4-hydroxycinnamic acid, may be deposited onto a reference analysis zone, for example. Prior to ionizing the characterization zones, the calibrant is ionized on this reference analysis zone. The m/z ratios of the ionized molecules of the calibrant then act as standards in order to enable the instrument to be used to measure the masses appropriately.

The method in accordance with the invention may in particular be employed to detect resistance due to the capacity of a microorganism to secrete an enzyme that is known to degrade antibiotics of the beta-lactam type, and in particular selected from penicillinases, cephalosporinases, cephamycinases, and carbapenemases. The invention is also suitable for detecting other resistance phenomena based on a degradation or an enzymatic modification causing a change in the mass of the antimicrobial agent. By way of example, it is possible to mention resistance mechanisms such as the degradation of macrolides by esterases or the degradation of fosfomycin by epoxidases, the acetylation of aminosides, chloramphenicol or indeed of streptogramins, the phosphorylation of aminosides, of macrolides, of rifampicin, and of peptide antibiotics, the hydroxylation of tetracyclin, the adenylation of aminosides and of lincosamides, ADP-ribosylation of rifampicin, and the glycosylation of macrolides and of rifampicin.

The term “degradation product” includes any product corresponding to a modification of the chemical structure of the antimicrobial agent due to the action of the microorganism present. In addition to the degradation and enzymatic modification mechanisms mentioned above, it may also involve adding a group that is detectable in MALDI-TOF that inactivates the antimicrobial agent or that prevents it from binding to its target.

A method of this type for the detection of resistance may be carried out with pre-functionalized MALDI plates with the help of commercially available MALDI-TOF instruments. Only the calibration and the interpretation steps need to be adapted in order to enable resistance to be detected. Detecting resistance to antimicrobial agents, and in particular rapidly determining resistance to a given antibiotic in routine manner, which may be vital in many clinical cases, is now possible in the context of the invention.

The characterization method in accordance with the invention, which can be used to identify the presence of a microorganism that is resistant to antibiotics in a very short length of time is of particular interest for rapid diagnosis. This is particularly true for detecting carbapenemase-producing enterobacteria (CPE). The method in accordance with the invention can be used to carry out rapid tests in a hospital environment in order to adapt the antibiotic treatment that is administered in a rapid manner.

### Identification of a Microorganism

The microorganisms that may be identified by the method of the invention are all types of microorganisms, pathogenic or otherwise, encountered both in industry and in a clinical situation, which may present resistance phenomena to antimicrobial agents. They may be, and are preferably bacteria, molds, yeasts, or parasites. The invention is of particular application to the study of bacteria. Examples of microorganisms of this type that may be mentioned are Gram-positive, Gram-negative and Mycobacteria. Examples of genuses of Gram-negative bacteria that may be mentioned are: Pseudomonas, Escherichia, Salmonella, Shigella, Enterobacter, Klebsiella, Serratia, Proteus, Acinetobacter, Citrobacter, Aeromonas, Stenotrophomonas, Morganella, Enterococcus, and Providencia, and in particular Escherichia coli; Enterobacter cloacae, Enterobacter aerogenes, Citrobacter sp., Klebsiella pneumoniae, Klebsiella oxytoca, Pseudomonas aeruginosa, Providencia rettgeri, Pseudomonas putida, Stenotrophomonas maltophilia, Acinetobacter baumank Comamonas sp., Aeromonas sp., Morganella morganii, Enterococcus sp., Proteus mirabilis, Salmonella senftenberg, Serratia marcescens, Salmonella typhimurium etc. Examples of genuses of Gram-positive bacteria that may be mentioned are: Enterococcus, Streptococcus, Staphylococcus, Bacillus, Listeria, and Clostridium.

Reference spectra obtained by MALDI-TOF for microorganisms of this type corresponding to their major proteins are available and stored in the databases bundled with commercial MALDI-TOF instruments, allowing the presence of such microorganisms to be identified by comparison.

The identification of the presence of a population of a microorganism may thus comprise the following steps:

a2) providing a reference mass spectrum, for at least one microorganism, and usually for a series of microorganisms;

b2) applying ionization to the population of microorganism(s) and the antimicrobial agent deposited on the analysis zone and in the presence of the matrix (corresponding to a characterization zone in accordance with the invention);

c2) acquiring a mass spectrum obtained following this ionization, in the range of masses of interest for the identification of the microorganism; and

d2) comparing the mass spectrum obtained in step c2) with the reference spectrum and deducing therefrom the family, the genus, or, as is preferable, the species of at least one microorganism.

When identifying microorganisms, calibration is carried out in a range of masses corresponding to high masses, typically in the range 2000 Da to 20000 Da, and preferably in the range 3000 Da to 17000 Da. The mass spectrum obtained in step c2) is also included in this range of masses.

The calibration may be carried out by placing a population of a reference microorganism in a reference analysis zone present on the plate and analyzing it by MALDI-TOF. By way of example, a reference microorganism of this type may be an E. coli bacterium. For this calibration, it is possible to use procedures described for carrying out the standard identification of microorganisms in the instruction manuals for commercial MALDI-TOF instruments such as the VITEK® MS instrument marketed by bioMérieux.

It is possible to use two reference analysis zones for the calibration: one to identify the microorganism and another to characterize the resistance. It is also possible to use the same reference zone to carry out both calibrations. In such circumstances, the reference zone should be functionalized with the antimicrobial agent of resistance that is to be studied.

In the context of the invention, in preferred but non essential manner, when the following two characterizations: detecting the resistance and identifying a microorganism; are both to be carried out, resistance is detected afterwards, i.e. the ionization and analysis steps necessary for detecting resistance are carried out on the characterization zone after the steps necessary for identifying the microorganism.

When two characterizations are performed on one and the same characterization zone, this may be done without removing the analysis plate from the mass spectrometer used for the MALDI-TOF analysis. This double characterization may thus comprise the following steps:

a3) providing a reference mass spectrum 1 for at least one microorganism and usually for a series of microorganisms as well as a mass spectrum 2 for the antimicrobial agent and/or for its degradation products;

b3) calibrating the mass spectrometer used by means of the calibrant used for the identification and that has already been deposited on the reference analysis zone;

c3) applying ionization to the population of microorganism(s) and the antimicrobial agent deposited on the analysis zone and in the presence of the matrix (corresponding to a characterization zone in accordance with the invention);

d3) acquiring a mass spectrum obtained following this ionization, in the range of masses of interest for identifying the microorganism;

e3) calibrating the mass spectrometer used a second time by means of the calibrant used to detect the resistance and that has already been deposited on a reference analysis zone;

f3) applying ionization again to the population of microorganism(s) and the antimicrobial agent deposited on the analysis zone and brought into the presence of the matrix (corresponding to a characterization zone in accordance with the invention);
g3) acquiring a mass spectrum obtained following this ionization, in the range of masses of interest for determining the resistance;
h3) comparing the mass spectrum obtained in step d3) with the reference spectrum or spectra 1 and deducing therefrom the family, the genus, or, as is preferable, the species of at least one microorganism that is present; and
i3) comparing the mass spectrum obtained in step g3) with the reference spectrum 2 and deducing therefrom the presence or otherwise of a resistance.

The steps c3) and f3) are therefore carried out on one and the same characterization zone and thus on one and the same population of microorganism(s), which thus means that the preparation and manipulation steps can be reduced.

In the context of the invention, the same population of microorganism(s) and a single deposit thereof on a single analysis zone are used in order to produce a characterization zone to which two ionizations are applied in succession in order to obtain the two characterizations (characterization of a phenomenon of resistance of a microorganism to an antimicrobial agent and identification of a microorganism).

In fact, it has unexpectedly been shown that the presence of the antimicrobial agent and carrying out a prior incubation step necessary for detecting a resistance phenomenon, do not in any way impede the possibility of identifying the microorganism.

The comparison steps h3) and i3) may be carried out at the end of the method or after each acquisition in question.

Preferably, the deposited population corresponds to a population of a single microorganism and the two characterizations—detection of the resistance and identification of a microorganism—relate to the same microorganism.

In conclusion, the invention can be used in order to:
Rapidly obtain information from one and the same characterization zone and in particular both characterizing a phenomenon of resistance to an antimicrobial agent and identifying a microorganism;
Simplify MALDI-TOF analysis methods without lengthy and tedious preparation of a sample before depositing it on a MALDI plate for characterizing a phenomenon of resistance to an antimicrobial agent;
Produce an optimum contact, directly on the MALDI plate, of the microorganism and the antimicrobial agent, sometimes having a positive impact on the sensitivity of detection of the resistance phenomenon;
Obtain a reduction in time and cost as regards consumables and manual operations in order to carry out a characterization of a phenomenon of resistance to an antimicrobial agent by MALDI-TOF; and
Obtain better traceability and better management of the samples to be analyzed, given that no previous incubation in the presence of an antimicrobial agent is necessary before it is deposited on a MALDI plate.

The examples below are intended to illustrate the invention but they are not limiting in any way. The analyses were carried out with the VITEK® MS instrument marketed by bioMérieux. The analyses were carried out in each of the examples below at the end of acquisition. The spectra were acquired over all of the characterization zones and then analyzed:
for identification, the spectrum was analyzed with the aid of the VITEK MS engine and the database V2.0.0 contained in the Spectra Identifier Version: 2.1.0 software;
for hydrolysis, the peaks of the spectra were visualized with the aid of Launchpad V2.8 acquisition software.

### Example 1

Preliminary experiments were carried out in order to show that it is possible, by MALDI-TOF, to detect antibiotic peaks of the beta-lactam type following deposition, on an analysis zone of a MALDI plate, of a drop of a solution containing the antibiotic mixed with an appropriate matrix for the MALDI technique. In the present experiment, an analysis zone of a MALDI plate (VITEK® MS disposable slides TO-430) was treated with a beta-lactam antibiotic and tested in order to evaluate the capacity of the antibiotics to be cleaved by a beta-lactamase. Ampicillin was used as the model for the antibiotic beta-lactam. The experiment was carried out by implementing the steps below:
2 μL of a solution of ampicillin in a concentration of 10 mg/mL in water was deposited in the form of a drop onto an analysis zone of the MALDI plate. The plate was incubated at 37° C., until the drop had dried out completely.
2 μL of a recombinant beta-lactamase (β-lactamase from Pseudomonas aeruginosa, Sigma-Aldrich batch L6170-550UN. CAS: 9073-60-3).
2 μL of recombinant β-lactamase at a concentration of 1 mg/mL in water was used diluted in water supplemented with zinc in the form of zinc sulfate (0.76 millimoles (mM)) to promote enzymatic activity, was deposited onto the dry analysis zone carrying ampicillin. A negative control was also carried out at the same time, by depositing, onto another analysis zone treated in the same manner with ampicillin, a drop of 2 μL of water with 0.76 mM of zinc, but without enzyme.
The plate was incubated at ambient temperature for one hour so that the enzymatic reaction could take place.
1 μL of a HCCA matrix, alpha-cyano 4-hydroxycinnamic acid (VITEK MS CHCA matrix, bioMérieux Ref: 411071), was deposited onto the analysis zones already containing the ampicillin with or without recombinant beta-lactamase.
MALDI-TOF mass spectrometry analysis was carried out with a VITEK® MS instrument using 2 μL of a mixture of HCCA and of pepMIX 6 (purchased from LaserBio Labs) used as a calibrant, which had been deposited on a reference analysis zone, to calibrate the instrument for low masses. This mixture was prepared by adding one volume of pepMix6 (10×) to 9 volumes of HCCA. The solution of pepMix6 (10×) was prepared in a diluent (trifluoroacetic acid, 0.01% in ultrapure water), in accordance with the manufacturer's recommendations. The peaks were acquired for a low range of masses of 200 Da to 1200 Da and the presence of molecules corresponding to the native molecule of ampicillin or to its degradation by-products following hydrolysis was studied.

All of the samples were analyzed on the plate in duplicate.

FIG. 2 plots the mass spectra obtained and shows that the ampicillin was not stable on the plate and that it was effectively degraded by the beta-lactamase after incubation. In fact, in FIG. 2, a major peak was observed at 368.09 m/z, corresponding to the hydrolyzed form of the ampicillin (mass calculated of the monosodium form of ampicillin corresponding to 368.4 m/z) and a complete loss of the native form at 372.09 m/z (mass calculated for the monosodium form of ampicillin corresponding to 372.4 m/z) on the top spectrum corresponding to the analysis zone with beta-lactamase. In contrast, for the negative control (bottom spectrum), the major peak was at 372.09 m/z, which corresponds to the monosodium native form of ampicillin. A little spontaneous hydrolysis of ampicillin was also observed in the negative control. This demonstrates that the enzymatic hydrolysis reaction of the beta-lactams can be detected by the MALDI-TOF technique, by using a method of preparing the characterization zone in accordance with the invention.

### Example 2

This experiment was carried out in order to study the degradation of ampicillin by various strains of bacteria after depositing colonies directly onto analysis zones carrying ampicillin, after depositing a solution thereof and drying, as described in Example 1.

The bacterial strains and their characteristics are presented in Table 1 below.

TABLE 1

| Bacterial | | | | |
|---|---|---|---|---|
| strain/ | | | | |
| reference | | MIC* of | | |
| number | Species | ampicillin | Phenotype | Virulence factor** |
| 1 | K. pneumoniae | 32 | resistant | — |
| 2 | E. coli | >128 | resistant | penicillinase |
| 3 | E. coli | >128 | resistant | TEM β-lactamase |
| 4 | E. coli | 4 | sensitive | no β-lactamase |
| | ATCC 25922 | | | |

*MIC: minimum inhibiting concentration of the strain
**Virulence factor: corresponds to the known secreted enzyme

The tests were implemented by carrying out the steps below:
2 μL of an aqueous solution of ampicillin in a concentration of 10 mg/mL, supplemented with zinc (0.76 mM), was deposited onto the analysis zones of the MALDI plate (same as that of Example 1).
The plate was incubated at 37° C., until the drops of the deposited solution had dried out completely.
For each bacterial strain, a portion of the colony obtained following growth for 24 h on gel medium was deposited in duplicate onto analysis zones carrying the ampicillin. Each spot was obtained as described in the VITEK® MS procedure for the identification of microorganisms.
The plate was incubated at ambient temperature (20-25° C.) for one hour in a moist atmosphere. To this end, a closed vessel containing water was used as an incubation chamber for the plate.
1 μL of a HCCA matrix was added to the analysis zones carrying both the antibiotic and bacteria.
A MALDI-TOF mass spectrum analysis was carried out with a VITEK® MS instrument, with the plate being put under vacuum after being placed in the chamber of the instrument, by carrying out the following two steps:
   first acquisition of the spectrum of the characterization zones, after calibrating the instrument from a reference zone carrying a deposited strain of E. coli(E-cal);
   second acquisition of the same characterization zones after calibrating the instrument on small masses with pepMIX 6 as the calibrant; this second acquisition was carried out immediately after the first, without breaking the vacuum of the chamber, and also without removing the plate from the instrument.
The data was collected and compared with data contained in the database in order to identify the species.
The peaks were viewed for a range of low masses in order to detect the native or hydrolyzed form of the antibiotic.

All of the samples were analyzed on the plate in duplicate.

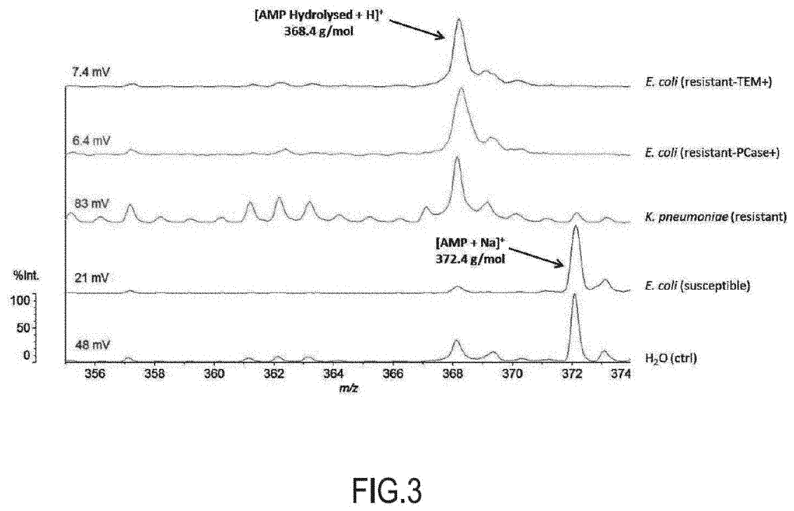

FIG. 3 plots the mass spectra obtained during the second series of acquisitions and shows that the ampicillin-resistant strains were all capable of hydrolyzing ampicillin under the experimental conditions used, while no degradation of the ampicillin was observed in the case of the strain E. coli ATCC 25922, which is known to be sensitive to ampicillin. In fact, after the second acquisition in the low masses, a major peak at 372.15 m/z, corresponding to the native form of ampicillin, was observed for the sensitive strain and the negative control, while for all of the ampicillin-resistant strains, the major peak was located at 368.15 m/z, corresponding to the hydrolyzed form of ampicillin and to the disappearance of the native form.

After the first acquisition and obtaining the corresponding spectra, all of the strains of bacteria could be identified with a high level of confidence.

As can be seen in Table 2 below, the bacteria could be identified correctly by MALDI-TOF from the first series of acquisitions for all of the characterization zones, with a level of confidence that lies in the range 99.9% to 100%, showing that the experimental conditions were also able to discriminate between the different species.

TABLE 2

| | | | Probability after |
|---|---|---|---|
| Bacterial strain/ | No of peaks used | | comparison of |
| reference number | per identification | Species | reference spectra |
| 1 | 109 | K. pneumoniae | 99.9 |
| 1 | 93 | K. pneumoniae | 100 |
| 2 | 95 | E. coli | 100 |
| 2 | 103 | E. coli | 100 |
| 3 | 91 | E. coli | 99.9 |
| 3 | 101 | E. coli | 99.9 |
| 4 | 94 | E. coli | 99.9 |
| 4 | 112 | E. coli | 99.9 |

### Example 3

Other tests were carried out in order to demonstrate that identification of the species and detection of the production of carbapenemase was possible from one and the same characterization zone. To this end, faropenem was used as a model of the antibiotic carbapene, and an identical protocol to that employed in Example 2 was used. A solution of faropenem was prepared with a concentration of faropenem of 0.5 mg/mL.

The bacterial strains used are detailed in Table 3 below.

TABLE 3

| Bacterial strain/ | | | |
|---|---|---|---|
| reference number | Species | Phenotype | Virulence factor |
| 5 | S. marcescens | resistant | IMP-1 |
| | | | carbapenemase |
| 6 | E. coli | sensitive | no β-lactamase |
| | ATCC 25922 | | |

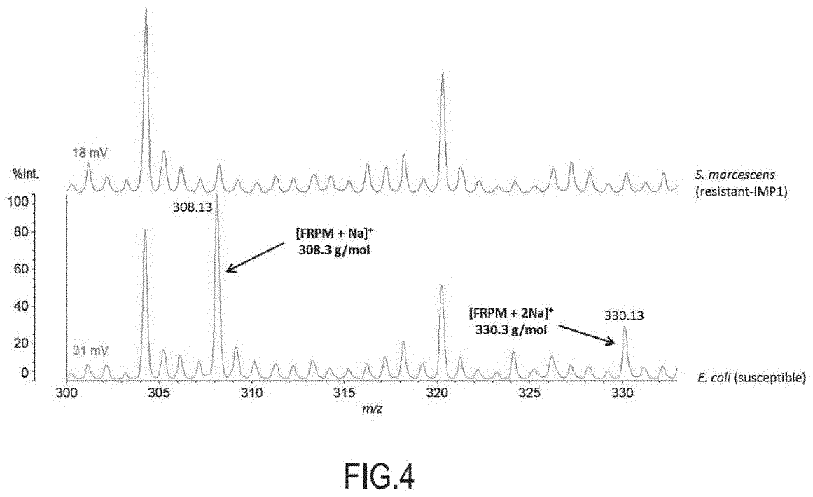

FIG. 4 plots the mass spectra obtained during the second series of acquisitions and shows that carbapenemase activity could be detected by MALDI-TOF under these conditions by means of the second series of acquisitions. Although the hydrolyzed forms of faropenem were not detected in this experiment, it was possible to demonstrate the loss of the two native forms of faropenem, due to the production of carbapenemase by the bacteria.

After acquisition in the low masses, for the sensitive strain a major peak was observed at 308.13 m/z with a minor peak at 330.13 m/z, respectively corresponding to the two native forms of faropenem (corresponding to calculated masses of 308.3 and 330.3 m/z). These two peaks were lost when faropenem had been incubated with the S. marcescens strain producing the carbapenemase IMP-1.

After the first acquisition and obtaining the spectra, both strains could be identified correctly with a high level of confidence. In similar manner to Example 2, the presence of faropenem in the characterization zones did not affect the capacity of MALDI-TOF to identify the species and the possibility of discriminating the E. coli type bacteria from bacteria of the marcescens type, as shown by the results obtained with the first acquisition series presented in Table 4.

TABLE 4

| | | | Probability after |
|---|---|---|---|
| Bacterial strain/ | No of peaks used | | comparison of |
| reference number | per identification | Species | reference spectra |
| 5 | 100 | S. marcescens | 99.9 |
| 5 | 113 | S. marcescens | 99.9 |
| 6 | 109 | E. coli | 99.9 |
| 6 | 101 | E. coli | 99.9 |

### Example 4

This test was carried out in order to increase the adhesion of the antibiotics to the analysis zone of the MALDI plate. In fact, drying a solution of antibiotic deposited on the plate resulted in the formation of a film that adhered weakly to the surface. Heptakis(2,6-di-O-methyl)-β-cyclodextrin (Heptakis, Sigma-Aldrich ref H0513) was used to bond the faropenem to the surface of the MALDI plate. Apart from its adhesion properties, the choice of using Heptakis was prompted by its molecular weight of 1331.36 grams per mole (g·mol⁻¹), which did not interfere with the mass peaks for faropenem or for its hydrolyzed products. During this experiment, two mass ratios [Heptakis]/[faropenem] were tested in order to evaluate the adhesion of dry faropenem and the impact of the Heptakis on the detection of native peaks and hydrolyzed peaks of faropenem.

The bacterial strains used are detailed in Table 5 below.

TABLE 5

| Bacterial strain/ | | | Virulence |
|---|---|---|---|
| reference number | Species | Phenotype | factor |
| 7 | K. pneumoniae | resistant | KPC |
| | | | carbapenemase |
| 8 | E. coli | sensitive | no β-lactamase |
| | ATCC 25922 | | (wild type) |

The tests were implemented by carrying out the steps below:
2 solutions containing a mixture of Heptakis and faropenem and 1 solution of faropenem without Heptakis were prepared in a NaCl buffer (0.45%) supplemented with zinc (zinc sulfate, 0.76 mM). The final concentrations of Heptakis and faropenem in these solutions were as follows:
   0 mg/mL of Heptakis and 1 mg/mL of faropenem;
   0.1 mg/mL of Heptakis and 1 mg/mL of faropenem (weight ratio 1:10);
   0.2 mg/mL of Heptakis and 1 mg/mL of faropenem (weight ratio 1:5);
2 μL of each solution was deposited onto the analysis zones of the MALDI plate (in accordance with that of Example 1);
the plate was incubated at ambient temperature, until the drops of the deposited solution had dried out completely;
to evaluate adhesion, each analysis zone functionalized with faropenem or with Heptakis/faropenem was scraped with the aid of an inoculation loop in order to mimic the deposition of a colony and a photo of the slide was taken;
in order to evaluate the effect of the concentration of Heptakis on detection of the faropenem peaks, a portion of the colony obtained following growth for 24 h on a gel medium was deposited in quadruplicate on the analysis zones carrying faropenem alone or mixtures of faropenem and Heptakis;
the plate was incubated at 37° C. for 2 h in a moist atmosphere;
1 μL of a HCCA matrix was added to the analysis zones also carrying antibiotic or Heptakis/antibiotic mixtures and bacteria;
analysis by MALDI-TOF mass spectrometry was carried out with a VITEK® MS instrument, in order to acquire low mass spectra;
the peaks of the native form at 308.3 m/z (faropenem+Na) and of the hydrolyzed form at 304.3 m/z (hydrolyzed faropenem+H) of faropenem, as well as the peak for HCCA at 212.03 m/z, were viewed;
for all of the test conditions, the intensities of the three peaks were recorded and the 308/212 and 304/212 ratios were calculated. The peak for HCCA at 212 m/z was used in this example as a control peak of unvarying intensity.

All of the samples were analyzed on the plate in quadruplicate.

FIG. 5 shows the appearance of the analysis zones (spots) before and after scraping with an inoculation loop. The photo shows a good dispersion, after scraping, of the Heptakis/faropenem mixture over the entire surface of the spot for [Heptakis]/[faropenem] ratios of 1/10 and 1/5, with good stability after scraping. The antibiotic that had dried without Heptakis was not very stable on the surface of the slide and the film was completely or partially detached during scraping with the inoculation loop.

FIG. 6 shows the variation in the ratios of the intensities of the peaks of native faropenem and hydrolyzed faropenem compared with the control peak of HCCA as a function of the [Heptakis]/[faropenem] ratios used when drying the antibiotic on the slide. The values represent the mean of four spots. For ratios of 1/10 and 1/5, detection was comparable to the condition without Heptakis, with an additional advantage of reproducibility in the presence of Heptakis. In fact, the substantial variability observed in the absence of Heptakis (large error bar) was due to the total or partial loss of the antibiotic on certain spots during deposition of the colony. Regarding the appearance of the hydrolyzed peak following incubation with the type KPC carbapenemase-producing strain, we observed the same phenomenon, i.e. good detection with [Heptakis]/[faropenem] ratios of 1/10 and 1/5.

The results of this experiment show that using Heptakis in suitable concentrations (0.1 mg/mL or 0.2 mg/mL for 1 mg/mL of faropenem) mean that the antibiotic can be stabilized on the slide for storage while ensuring optimized mixing with the bacterium during deposition of the colony. At these same concentrations, Heptakis can also be used to improve the reproducibility of the results between the spots and does not interfere with detecting the peaks, or indeed with the faropenem hydrolysis reaction.

### Example 5

This test was carried out in order to evaluate the possibility of carrying out the hydrolysis reaction on the functionalized analysis zone of the MALDI plate from a liquid deposit, i.e. a bacterial inoculum. In fact, the problem encountered during colony deposition is a lack of standardization as regards the quantity of microorganisms deposited and the variability of the deposit as a function of the operator. Thus, depositing a colony on a slide for a conventional MALDI-TOF identification application requires a technical training, and even that does not completely eliminate the risk of variability of the results due to heterogeneous deposits. In addition, a bacterial inoculum with a known concentration was applied to the functionalized analysis zones of the MALDI plate.

The bacterial strains used are set out in Table 5 above.

The tests were implemented by carrying out the steps below:
2 μL of a solution of faropenem at a concentration of 1 mg/mL prepared in a NaCl buffer (0.45%) supplemented with zinc (zinc sulfate, 0.76 mM) was deposited onto the analysis zones of the MALDI plate and dried at ambient temperature;
2 μL of bacterial inocula at concentrations of 3 McFarland or 6 McFarland were applied to the functionalized analysis zones in quadruplicate;
the plate was incubated at 37° C. for 2 h in a moist atmosphere;
1 μL of a HCCA matrix was added to the analysis zones carrying both the antibiotic and the bacteria;
MALDI-TOF mass spectrometry analysis was carried out with a VITEK® MS instrument, for two acquisitions, one in low mass spectra in order to observe the faropenem peaks, and another in high mass spectra for identification (in accordance with Example 2); and
the peaks for the native forms at 308.3 m/z (faropenem+Na) and at 330.3 m/z (faropenem+2Na) as well as the peak for the hydrolyzed form at 304.3 m/z (hydrolyzed faropenem+H) were viewed;
for all of the test conditions, the intensities of the three peaks were recorded and the 304/308 and 304/330 ratios were calculated in order to quantify the hydrolysis of faropenem.

All of the samples were analyzed on the plate in quadruplicate.

FIG. 7 plots the mass spectra obtained during the second series of acquisitions and shows that carbapenemase activity could be detected by MALDI-TOF following a deposit of bacterial inocula at a strength of 6 McFarland. In fact, it was possible to demonstrate the loss of the two native forms and the appearance of the hydrolyzed form of faropenem due to the production of carbapenemase by the bacteria.

After acquisition in the low masses, with the sensitive strain a major peak was observed at 308.03 m/z and a minor peak was observed at 330.02 m/z, corresponding respectively to the two native forms of faropenem (corresponding to calculated masses of 308.3 and 330.3 m/z). These two peaks were lost when the faropenem was incubated with the K. pneumoniae strain producing the carbapenemase KPC, and the appearance of a peak at 304.06 m/z was observed, corresponding to the hydrolyzed form of faropenem (calculated mass 304.03).

After the first acquisition and obtaining the spectra, the two strains could be correctly identified with a high level of confidence. The presence of faropenem in the characterization zones and depositing the inoculum did not affect the capacity for identifying the species by MALDI-TOF, and did not affect the possibility of discriminating bacteria of the E. coli type from bacteria of the K. pneumoniae type, as can be seen from the results obtained with the first series of acquisitions shown in Table 6.

TABLE 6

| | | | Probability after |
|---|---|---|---|
| Bacterial strain/ | No of peaks used | | comparison of |
| reference number | per identification | Species | reference spectra |
| 7 | 177 | K. pneumoniae | 91.2 |
| 8 | 149 | E. coli | 93 |

FIG. 8 shows the variation of the 304/308 and 304/330 intensity ratios as a function of the concentration of inoculum used for the 2 test strains. For the wild type strain that does not hydrolyze the antibiotic, no significant variation of the 2 ratios was observed regardless of the concentration of bacterial inoculum. For the strain-producing type KPC carbapenemase, a significant increase was observed in both ratios, which was proportional to the concentration of inoculum. This increase in the ratios resulted in a reduction in the intensity of the native peaks and in an increase in the intensity of the hydrolyzed peak, as shown in FIG. 7.

The results of this experiment show that the deposit of inoculum is also adapted to the hydrolysis reaction on the MALDI plate and to identification by MALDI-TOF mass spectrometry. In addition, the small error bars observed over a mean of four deposits suggest very good reproducibility of the results.

FIG. 9 represents an embodiment of a casing incorporating a MALDI plate that could be adapted to depositing a population of microorganisms in the liquid form. A MALDI plate slide with analysis zones that have already been covered with dried antibiotic was placed in a gallery inside which a plurality of wells (12 in the model shown) had been molded. Six wells aligned on the same axis contained an opacity control in the dehydrated form, and the other six wells were empty. The gallery containing the slide was itself placed in a cassette with a cover. For storage, this cassette could in particular be packaged in a hermetically sealed manner away from light and moisture.

The protocol for using this product may be described in the following steps:
remove the cover from the cassette;
fill the 12 wells with a volume of 50 μL to 100 μL of water or physiological buffer. Filling the six wells containing the opacity control causes turbid solutions to be formed;
prepare the inocula in the other six wells by adding a colony or a portion of a colony so as to obtain a turbidity comparable to the opacity control of the facing well;
deposit 2 μL of each inoculum onto the analysis zones carrying the antibiotic;
add water to the cassette with the aid of a pipette in order to generate a moist atmosphere;
replace the cover and incubate at 37° C. for 1 h to 2 h (or less);
add 1 μL of a HCCA matrix to the analysis zones carrying both the antibiotic and bacteria; and
recover the slide for analysis by MALDI-TOF mass spectrometry.

Adding the opacity control means that it is possible to avoid preparing the inoculum by using density or turbidity measuring apparatus, and can thus save time. Furthermore, measurement apparatus in routine use such as densitometers are adapted to large volumes (1 mL to 3 mL), thus requiring a large quantity of bacteria to be used.

The inoculum may also be prepared using a solid or liquid extract of bacteria obtained directly from a biological sample (e.g.: bacteria extracted from a hemoculture).

The embodiment shown in FIG. 9 allowed six different strains to be tested. This model could be adapted as a function of the quantity of routinely tested strains, in particular by increasing the number of wells.

### REFERENCES

Hooff, G. P., J. J. van Kampen, R. J. Meesters, A. van Belkum, W. H. Goessens and T. M. Luider (2012). “Characterization of beta-lactamase enzyme activity in bacterial lysates using MALDI-mass spectrometry.” J Proteome Res 11(1): 79-84.
Hrabak, J., V. Studentova, R. Walkova, H. Zemlickova, V. Jakubu, E. Chudackova, M. Gniadkowski, Y. Pfeifer, J. D. Perry, K. Wilkinson and T. Bergerova (2012). “Detection of NDM-1, VIM-1, KPC, OXA-48, and OXA-162 carbapenemases by matrix-assisted laser desorption ionization-time of flight mass spectrometry.” J Clin Microbiol 50(7): 2441-2443.
Hrabak, J., R. Walkova, V. Studentova, E. Chudackova and T. Bergerova (2011). “Carbapenemase activity detection by matrix-assisted laser desorption ionization-time of flight mass spectrometry.” J Clin Microbiol 49(9): 3222-3227.
Sparbier, K., S. Schubert, U. Weller, C. Boogen and M. Kostrzewa (2012). “Matrix-assisted laser desorption ionization-time of flight mass spectrometry-based functional assay for rapid detection of resistance against beta-lactam antibiotics.” J Clin Microbiol 50(3): 927-937.

1. A method of characterizing a population of a microorganism, the method comprising:
preparing a characterization zone of an analysis plate; followed by
characterizing the population of a microorganism,
in which:
preparing the said characterization zone comprises the following steps in succession:
a step of providing an analysis plate for a characterization by means of the the matrix-assisted laser desorption-ionization (MALDI) mass spectrometry technique, the plate comprising a functionalized analysis zone carrying an antimicrobial agent;
a step of depositing the population of a microorganism onto said functionalized analysis zone in contact with the antimicrobial agent;
an incubation step of preserving the analysis plate under conditions and for a sufficient time to allow the antimicrobial agent and the microorganism that is present to interact; and
a step of depositing a matrix that is suitable for the MALDI technique onto the functionalized analysis zone;
characterizing the population of a microorganism involves an analysis by mass spectrometry, during which, the population of the microorganism deposited on the characterization zone undergoes an ionization step by bombarding the analysis zone with a laser beam in accordance with the matrix-assisted laser desorption-ionization mass spectrometry (MALDI) technique and comprises:
identifying the family, genus, or species of a population of a microorganism deposited on the characterization zone by carrying out a first analysis by the matrix-assisted laser desorption-ionization (MALDI) mass spectrometry technique corresponding to a first step of ionizing the characterization zone, and by using a first calibration for the analysis; and
determining the possible presence on the characterization zone of a population of a microorganism that is resistant to the antimicrobial agent by carrying out a second analysis by mass spectrometry using a matrix-assisted laser desorption-ionization time of flight (MALDI-TOF) mass spectrometry technique corresponding to a second step of ionizing the same characterization zone, and by using a second calibration for the analysis.

2. The method according to claim 1, wherein a population of a single microorganism to be characterized is deposited.

3. The method according to claim 1, wherein the deposited population of a microorganism is prepared without a prior step of contact with an antimicrobial agent.

4. The method according to claim 1, wherein the population of a microorganism is obtained after a step of concentration, enrichment and/or purification, and/or corresponding to a colony, or to a fraction of a colony obtained, after growth on a suitable medium.

5. The method according to claim 1, wherein the antimicrobial agent is selected in a manner such as to allow the resistance due to the production of beta-lactamase to be identified.

6. The method according to claim 1, wherein the antimicrobial agent is an antibiotic selected from penicillins, cephalosporins, cephamycins, carbapenems, and monobactams.

7. The method according to claim 1, comprising a functionalization step in order to obtain the functionalized analysis zone carrying the antimicrobial agent.

8. The method according to claim 7, wherein the functionalization step is carried out by depositing an aqueous solution of the antimicrobial agent, followed by drying.

9. The method according to claim 1, wherein the functionalized analysis zone carries a single antimicrobial agent.

10. The method according to claim 1, wherein the analysis by mass spectrometry using the MALDI-TOF technique in order to conclude whether a population of a microorganism that is resistant to the antimicrobial agent is present on the characterization zone consists in verifying the presence of the antimicrobial agent, and/or of a degradation product of the antimicrobial agent.

11. The method according to claim 1, comprising identifying the family, genus, or species of a population of a microorganism, and determining whether the possible presence of the antimicrobial agent concerns the same microorganism.

12. The method according to claim 1, wherein the antimicrobial agent on the functionalized analysis zone was obtained by depositing an aqueous solution of the antimicrobial agent, followed by drying.

13. The method according to claim 1, wherein the functionalized analysis zone is in dried form.

14. The method according to claim 1, wherein the antimicrobial agent is immobilized on the functionalized analysis zone by electrostatic, ionic or covalent bonding.

15. The method according to claim 1, wherein the antimicrobial agent is immobilized on the functionalized analysis zone by affinity bonding, or by means of an adhesive agent.

16. The method according to claim 1, wherein the functionalized analysis zone carries from 0.04 g/m² to 4 g/m² of antimicrobial agent and the step of depositing the population of a microorganism leads to at least 10⁵ cfu deposited.
